(19) United States
(12) Patent Application Publication
(10) Pub. No.: US 2018/0177740 A1
(43) Pub. Date: Jun. 28, 2018

(54) NON-NATURAL FIBER OR FILAMENT WITH HERBAL RESIDUE AND METHOD OF MAKING THE SAME

(71) Applicant: ABHISHEK MANDAWEWALA, Mumbai (IN)

(72) Inventors: ABHISHEK MANDAWEWALA, MUMBAI (IN); PRAMOD KUMAR PATHAK, Silvassa (IN); PANKAJ VERMA, MUMBAI (IN)

(21) Appl. No.: 15/424,851

(22) Filed: Feb. 5, 2017

(30) Foreign Application Priority Data
Dec. 27, 2016 (IN) 201621044524

(51) Int. Cl. A61K 9/70; A61K 9/14; A61K 47/32; A61K 47/34; A61K 36/00; A61L 15/24; A61L 15/22; A61L 15/46; D02G 3/04; D02G 3/44; D01D 5/26

(52) U.S. Cl. CPC A61K 9/70; A61K 9/14; A61K 47/32; A61K 47/34; A61K 36/00; A61K 2236/15; A61L 15/22; A61L 15/46; D02G 3/04; D02G 3/449; D01D 5/26; A61L 15/24

(57) ABSTRACT

The non-natural fiber or filament composition comprises an herbal masterbatch, a base polymer powder or a polymer, and one or more additives such as coupling agent, UV stabilizer, antioxidant, Antistatic agent, colorants, pigments, and the like. The herbal masterbatch further comprises an extract of at-least a natural or herbal product. The herbal masterbatch further provides multiple functional benefits such as anti-microbial, anti-odor, cooling effect etc. to the non-natural fiber or filament. Further, the method of preparing the natural or herbal masterbatch, comprises the steps of grinding at-least one of an herbal or natural products to produce an herbal extract having a particle size of less than or equal to 100 microns, or preferably less than 50 microns, or more preferably less than 25 microns, and mixing or blending the herbal extract with a coupling agent, a base polymer, and one or more from the additives such as Coupling agent, UV stabilizer, antioxidant, Antistatic agent, colorants, pigments, and the like, in a high speed mixer. In one embodiment, drawing a yarn or filament or fiber from the herbal masterbatch resulted into the non-natural fiber or filament. In another embodiment, the blended mix is extruded together through conventional spinning processes known in the art to form the non-natural fiber or filament having denier range between 0.3 DPF to 30 DPF exhibiting multiple functional benefits.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### TECHNICAL FIELD

Embodiments described herein relate to a non-natural fiber or filament composition comprising an herbal residue. The embodiments further relate to an herbal masterbatch made with the use of natural and/or herbal products, polymers and additives. More particularly the embodiments described herein relate to the masterbatch for use in manufacturing of chemical-free non-natural fiber or filament having additional beneficial properties such as anti-microbial & anti-odor behavior.

### BACKGROUND

Textile industry has conventionally been using both natural and synthetic fibers for manufacture of various products. Each having their own advantages and shortcomings related to strength, comfort, softness, longevity and the like. With ever-increasing demand to provide enhanced quality fibers manufacturers apart from enhancing traditional properties, have started incorporating more functional elements to fibers to provide better comfort, freshness, and hygiene to users. In pursuit of meeting demands of modern hygienic lifestyle, there have been several attempts to add a variety of chemical agents to fabrics providing them with anti-microbial properties. These agents are normally applied on the surface to impart anti-microbial capabilities to the fabrics during finishing stage. However, items made with such fabrics are unable to retain anti-microbial capabilities for long and as the compounds are washed-off during washings or ultra-violet exposure. A significant reason that these fibers are not able to provide long-term anti-microbial effects lies on the fact that these components are added to the fiber during the fabric finishing stage through the use of various finishing chemicals. Moreover, there is always a concern regarding use of such chemical agents and their potential after-effects on human body.

One way of overcoming this problem is through use of a masterbatch during the initial stages of fiber production, thereby adding longevity to the functional benefit to the fabrics made from such fibers. A masterbatch (MB) is an additive used for obtaining multiple functional benefits to the synthetic fibers. However, chemical masterbatches are expensive, and a different additive is required to obtain each different function. Therefore, there exists a need for innovation in relation to this technology to overcome the shortcomings prevalent in the market.

The present invention provides a non-natural fiber or filament offering functional benefits such as anti-microbial, anti-odor, cooling effect, and the like and method of preparing the same. The present invention also provides a natural and herbal masterbatch to be used in the preparation of the non-natural fiber or filaments and method of preparing the same. An objective of the present invention is to use inexpensive natural and herbal products such as neem, babool, etc. in production of non-natural fiber or filaments to provide beneficial properties such as anti-microbial, anti-odor, cooling effect and the like.

### SUMMARY

This Summary is provided to introduce a selection of concepts in a simplified form that are further described below in the Detailed Description. This Summary is not intended to identify key features or essential features of the claimed subject matter, nor it is intended to be used to limit the scope of the claimed subject matter.

An object of the present invention is to provide a non-natural fiber or filament composition and/or a non-natural fiber or filament, exhibiting functional benefits such as anti-microbial, anti-odor, cooling effect, and the like.

An object of the present invention is to provide a method of manufacturing a non-natural fiber or filament composition and/or a non-natural fiber or filament, exhibiting functional benefits such as anti-microbial, anti-odor, cooling effect, and the like.

An object of the present invention is to use inexpensive natural and herbal products or processed herbal products in the manufacture of non-natural fiber or filaments to exhibit functional benefits such as anti-microbial, anti-odor, cooling effect and the like.

An object of the present invention is to manufacture a non-natural fiber or filament exhibiting functional benefits comprising a natural and herbal masterbatch composition.

Another object of the present invention is to manufacture a natural and herbal masterbatch composition, comprising natural and herbal products, polymers and additives such as coupling agent, UV stabilizer, antioxidant, antistatic agent or colorants in high speed mixer or mixers thereof.

Another object of the present invention is to manufacture non-natural fiber or filaments without the use of chemical agents for providing functionally beneficial properties such as anti-microbial, anti-odor, cooling effect and the like.

A yet another object of the present invention is to introduce a plurality of functional benefits to a non-natural fiber for users.

The present invention further provides a fabric comprising the non-natural fiber or filament composition and/or a non-natural fiber or filament of the present invention having the non-natural fiber or filament having denier range between 0.3 DPF to 30 DPF.

### DETAILED DESCRIPTION

The presently disclosed subject matter is described with specificity to meet statutory requirements. However, the description itself is not intended to limit the scope of this patent. Rather, the inventors have contemplated that the claimed subject matter might also be embodied in other ways, to include different steps or elements similar to the ones described in this document, in conjunction with other present or future technologies.

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise.

Furthermore, the use of the term “including”, as well as other forms, such as “includes” and “included”, is not limiting. Also, terms such as “element” or “component” encompass both elements and components comprising one unit and elements or components that comprise more than one unit unless specifically stated otherwise.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

According to an aspect of the present disclosure, there is provided a non-natural fiber or filament composition. The non-natural fiber or filament composition comprises an herbal masterbatch. The herbal masterbatch further comprises an herbal extract (an extract of at-least one natural or herbal products) of particle size <50 microns, a polymer powder or a polymer, and one or more from the additives such as coupling agent, UV stabilizer, antioxidant, Antistatic agent, colorants, pigments, and the like. The herbal masterbatch may further comprises a processed extract or a raw extract or a mixture of processed and raw extract of at-least one natural or herbal products. The herbal masterbatch further provides multiple functional benefits such as anti-microbial, anti-odor, cooling effect, etc. to the non-natural fiber or filament.

Further, the natural or herbal products used in making of herbal masterbatch composition preferably comprises of plant extracts or processed plant extract or a mixture of the same. The plant or processed plant extracts used majorly comprises of plants that have medicinal, savory, aromatic, anti-microbial or anti-odor qualities. The plants comprising of these properties includes plants for example, Azadirachta indica, Curcuma longa, Prickly acacia, Mentha piperita, Margosa, Oolong tea, oregano, magnolia, cranberry, rosemary, Camellia, morin, Garcinia mangostana L., Jabara, Juglans regia, Zanthoxylum alantum, Mimusops elengi, Hibiscus abelmoschus, Ayurvedic, Carapa procera, Khaya senegalensis, Salvadora persica, Cucurbitaceae (Citrullus colocynthis) and mixtures thereof.

Any suitable plant extract can be used so long as it enhances the desired effects and other suitable plants for this purpose include but not limited to Acacia catechu, Acacia nilotica, Achyrathes aspera, Aristolochia bracteolate, Cinnamomum camphora, Cinnamomum verum, Curcuma longa, Eucalyptus globulus, Ficus bengalensis, Juglans regia, Madhuca longifolia, Mimusops elengi, Ocimum sanctum, Oolonga tea, Piper betel leaves, Piper longum, Piper nigrum, Potentilla fulgens, Syzygium aromaticum, Spilanthes calva, Vaccinium macrocarpon, Zanthoxylum armatum, and mixtures thereof.

Additional extracts can be selected from one or more plants of the following genera: Zingiberaceae, Mentha, Origanum Thymus, Lavandula, Salvia, Melissa, Cuminum, Petroselinum, Calendula, Tagetes, Boswellia, Sambucus, Copaifera, Curcuma, Allium, Symphytum, Euterpe, Sophora, Rheum, Fagopyrum, Camellia, Coptis, Hydrastis, Mahonia, Phellodendron, Berberis, Xanthorhiza, Lonicera, Vaccinium, Cinnamomum, Vitis, Terminalia, Pinus, Albizia, Melia, Salvadora, Paullinia, Piper, Syzygium, Commiphora, Juglans, Scutellaria, and Magnolia.

In one of the embodiments, the herbal masterbatch is in the form of powder or liquid or both. The reduction of herbal masterbatch in the form of powder is done as a process of pre-processing of at least one natural or herbal product. Further, the average size of powder is reduced to less than 100 microns or preferably less than 50 microns or more preferably less than 25 microns.

In one of the embodiments, the non-natural fiber or filament comprises the herbal extract, the coupling agent, the polymer, and one or more from the additives such as coupling agent, UV stabilizer, antioxidant, Antistatic agent, colorants, pigments, and the like, in a pre-defined ratio. The pre-defined ratio is 0.1-30% by weight of the at-least one of natural or herbal products, 0.1-10% by weight of the coupling agent and 50-99% wt. of the base polymer. Preferably, the pre-defined ratio is 0.1-25% by weight of the at-least one of natural or herbal products, 0.1-5% by weight of the coupling agent and 70-99% by weight of the base polymer and more preferably the pre-defined ratio is 0.1-5% by weight of the at-least one of natural or herbal products, 1-3%A by weight of the coupling agent and 80-98% by weight of the base polymer.

In one of the embodiments, the non-natural fiber or filament (final fiber) having denier range between 0.3 DPF to 30 DPF.

In one of the embodiments, the coupling agent is also added to the composition where the coupling agent aids in adhesion of the herbal extract and the polymer. It is to be appreciated that the coupling agents described herein are exemplary in nature, and any suitable coupling agent known to those of ordinary skill in the art may be utilized in any of the exemplary embodiments described or otherwise suggested herein. In some exemplary embodiments, the coupling agent, or coupling agents, may be Silane, Epoxysilane or Organosilane coupling agents. Besides their role of coupling the surface of the reinforcement fibers and the surrounding matrix, coupling agents also function to reduce the level of fuzz, or broken fiber filaments, during subsequent processing.

In one of the embodiments, the polymer or the base polymer is a thermoplastic polymer resin preferably of the polyester family selected from a group of PET, PBT, PTT, PLA, PP, Polyamide or a mixer thereof.

According to another aspect of the invention, there is provided a method of preparing an herbal masterbatch for producing a non-natural fiber or filament exhibiting functional benefits such as anti-microbial, anti-odor, cooling effect, and the like. The method comprises the steps of preparing a natural or herbal masterbatch and drawing a yarn or filament or fiber from the masterbatch to manufacture non-natural fiber or filament, wherein the amount of herbal extract in the non-natural fiber or filament (final fiber) is in a range of 0.1-30% by weight, or preferably is in a range of 0.1-25% by weight, or more preferably is in a range 0.1-5% by weight.

Further, the method of preparing the natural or herbal masterbatch, comprises the steps of grinding (or processing) at-least one of a herbal or natural products to produce a herbal extract having an average particle size of less than or equal to 100 microns, or preferably less than 50 microns, or more preferably less than 25 microns, and mixing or blending the herbal extract with a coupling agent, a polymer powder, and one or more from the additives such as Coupling agent, UV stabilizer, antioxidant, Antistatic agent, colorants, pigments, and the like, in a high speed mixer.

The class of materials used as UV stabilizer includes but is not limited to: ultraviolet absorbers, quenchers, and hindered amine light stabilizers. These compounds are used to prevent degradation of the polymer due to solar ultraviolet radiation exposure.

In one of the embodiments, the non-natural fiber or filament composition comprising the herbal masterbatch and the polymer is extruded together through conventional spinning processes known in the art to form the non-natural fiber or filament. In one of the exemplary embodiments, the blended mix may be extruded through twin screw extruder followed by melt blending to produce fibers or filaments having denier range between 0.3 DPF to 30 DPF.

According to one another aspect of the invention, there is provided a method of manufacturing a non-natural fiber or filament exhibiting functional benefits, comprising the steps of: preparing a natural or herbal masterbatch, blending or re-mixing the herbal masterbatch, the polymer, and one or more from the additives such as coupling agent, UV stabilizer, antioxidant, Antistatic agent, colorants, pigments, and the like to produce a masterbatch; and extruding the produced masterbatch through conventional spinning processes known in the art to manufacture non-natural fiber, wherein the amount of herbal extract in the extruded non-natural fiber is in a range of 0.1-30% by weight, or preferably is in a range of 0.1-25% by weight, or more preferably is in a range 0.1-5% by weight.

In one of the exemplary embodiments, the herbal masterbatch may mixed with at-least one other masterbatch and extruded to introduce a plurality of functional benefits to a non-natural fiber for users. The other masterbatch may be primarily a color masterbatch, anti-UV masterbatch, anti-static masterbatch and the like. In one of the embodiments, a single herbal masterbatch may be used to impart multiple functional benefits to the non-natural fiber or filament.

In one of the embodiments, the non-natural fiber or filament made using herbal masterbatch is a filament yarn or staple fiber.

In one of the embodiments, the non-natural fiber or filament made using herbal masterbatch is used to manufacturing of knitted, woven or non-woven fabrics.

In one of the embodiments, the non-natural fiber or filament made using herbal masterbatch is relatively inexpensive than a chemical added non-natural fiber or filament. The difference in cost may be attributed to the use of natural or herbal products instead of chemicals.

While the embodiments have been described in connection with the preferred embodiments of the various figures, it is to be understood that other similar embodiments may be used or modifications and additions may be made to the described embodiment for performing the same function without deviating therefrom. Therefore, the disclosed embodiments should not be limited to any single embodiment, but rather should be construed in breadth and scope in accordance with the appended claims.

We claim:

1. A non-natural fiber or filament composition comprises:
a. an herbal masterbatch, wherein the herbal masterbatch comprises:
i. an herbal extract, wherein the herbal extract comprises an extract of at-least one natural or herbal products of particle size <50 microns;
ii. at-least one coupling agent/comfortabiliser; and
iii. a polymer; and
b. a base polymer;
wherein the herbal masterbatch and the base polymer is extruded together to form the non-natural fiber having at-least one functional benefits such as anti-microbial, anti-odor, cooling effect etc; and,
wherein the amount by weight of herbal extract in the final fiber could be between 0.1-5%.

2. The non-natural fiber or filament composition as claimed in claim 1 is used to manufacturing of knitted, woven or non-woven fabrics.

3. The non-natural fiber or filament composition as claimed in claim 1, wherein the non-natural fiber or filament having denier range between 0.3 DPF to 30 DPF.

4. The natural or herbal products as claimed in claim 1, wherein the natural or herbal products are plant extracts or processed plant extracts or a mixture of the same.

5. The herbal extract as claimed in claim 1 is in a form of powder or liquid or both.

6. The powder as claimed in claim 3, wherein the powder is manufactured by processing the at-least one of natural or herbal products to an average size of less than 50 microns.

7. The non-natural fiber or filament as claimed in claim 1 comprises 0.1-5 parts by weight of the at-least a natural or herbal products.

8. The base polymer as claimed in claim 1, wherein the polymer or the base polymer is selected from a group of PET, PBT, PTT, PLA , PP, Polyamide or a mixer thereof.

9. A method of manufacturing anti-microbial, anti-odor non-natural fiber or filament comprising the steps of:
a. preparing a natural or herbal masterbatch , wherein the herbal masterbatch preparation comprising the steps of:
i. grinding at-least one herbal or natural products to produce a herbal extract having an extracted particle size of less than or equal to 50 microns; and
ii. mixing or blending the herbal extract, a coupling agent and a polymer to produce a masterbatch, wherein the ratio of the herbal extract to the base polymer is 30:70 by wt. in the prepared natural or herbal masterbatch; and
b. drawing a yarn or filament or fiber from the masterbatch wherein the amount of herbal extract in the final non-natural fiber or filament is between 0.1-5% by weight.

10. A method of manufacturing a natural or herbal masterbatch comprising the steps of:
a. grinding at-least one herbal or natural products to produce an herbal extract having an average extracted particle size of less than or equal to 50 microns; and
b. mixing or blending the herbal extract, a polymer and additives to manufacture natural or herbal masterbatch.

11. The method claimed in claim 9, wherein the additives comprise at-least a coupling agent, UV stabilizer, antioxidant, antistatic agent or colorants in high speed mixer or mixers thereof.

12. The method claimed in claim 9, wherein the natural or herbal extract are preferably plant extracts.

13. The method claimed in claim 9, wherein the manufactured non-natural fiber comprises at-least one functional benefit such as but not limited to anti-microbial, anti-odor, cooling effect, durability etc.

14. The method claimed in claim 9, the wherein manufactured non-natural fiber non- [symbol] is a filament yarn or staple fiber.

15. The method claimed in claim 9, wherein the coupling agent is used to couple the natural or herbal extract and the base polymer.

16. The method claimed in claim 9, wherein the polymer or the base polymer is selected from a group of PET, PBT, PTT, PLA, PP, Polyamide or a mixer thereof.

17. The method as claimed in claim 9 uses a single herbal masterbatch for obtaining multiple functional benefits to the non-natural fiber.

18. The method claimed in claim 10, wherein the herbal masterbatch is mixed with at-least another masterbatch and extruded to introduce a plurality of functional benefits to a non-natural fiber for users.
